(19) United States
(12) Patent Application Publication
(10) Pub. No.: US 2017/0312131 A1
(43) Pub. Date: Nov. 2, 2017

(54) VITRECTOMY APPARATUS, SYSTEM AND METHOD

(71) Applicant: H.S. International Corp., Concord, CA (US)

(72) Inventor: Madan Maholtra, Concord, CA (US)

(21) Appl. No.: 15/140,468

(22) Filed: Apr. 27, 2016

(51) Int. Cl. A61F 9/007

(52) U.S. Cl. CPC A61F 9/00763

(57) ABSTRACT

A vitrectomy cutter apparatus and method. The apparatus (and method) is efficient in facilitating removal of vitreous humor during intraocular surgery such as a vitrectomy procedure for the human eye. The apparatus includes an exterior cylindrical member with ports substantially separately and/or oppositely disposed from each other. An interior cylindrical member reciprocates within the exterior cylindrical member to cut vitreous received via the separately disposed ports. The interior cylindrical member includes a funnel-shaped distal end and a spiral cut axially to reduce friction between the external and internal cylindrical members.

### BACKGROUND

The present disclosure relates generally to vitrectomy probes and surgical instruments and more specifically to a vitrectomy apparatus, system and method.

Around the world, roughly 250 million people may have some kind of vision impairment that requires removal of vitreous humor from the eye. Vitreous humor also herein referred to as vitreous is a complex and fibrous gel-like substance that fills about 80 percent of the eye and helps to maintain the eye's round shape.

Vitreous removal is accomplished via vitrectomy, a surgical procedure for the eye that involves the placement of ports in the eye through which various instruments can be passed. A vitrectomy cutter, for example, is passed through one of the ports to cut and remove vitreous from the eye. As is then apparent, given the importance of the human eye, the procedure must be performed optimally with instruments that facilitate vitrectomy and minimize trauma that can arise during this surgical procedure.

It is within the aforementioned context that a need for the present invention has arisen. Thus, there is a need to address one or more of the foregoing disadvantages of conventional systems and methods, and the present invention meets this need.

### BRIEF SUMMARY OF THE INVENTION

Various aspects of an apparatus, method and system for a vitrectomy cutter can be found in exemplary embodiments of the present invention.

In one embodiment, the apparatus includes an exterior cylindrical member that has two substantially oppositely disposed or separated sets of vitreous ports. During a vitrectomy procedure for a human eye, the vitreous ports are configured to receive vitreous humor that can then be severed and aspirated. In one embodiment, a first set of vitreous ports may be structured to have at least three ports. A second set of vitreous ports may also be structured to have a least three separate or oppositely disposed ports.

The exterior cylindrical member includes an open and a closed end, where the vitreous ports are located. The apparatus also includes an interior cylindrical member that reciprocates axially within the exterior cylindrical member. A cutting edge is formed on an open distal end of the interior cylindrical member to cut and aspirate vitreous humor from the first and second sets of vitreous ports.

In an embodiment, the cutting edge of the interior cylindrical member is flared or structured to have a funnel shape. The cutting edge may also be tapered (from a proximal to distal direction). In another embodiment, an electrolyte layer is disposed on an exterior surface of the interior cylindrical member, the electrolyte layer being configured to harden and sharpen the cutting edge of the interior cylindrical member.

In another embodiment, a spiral slot is cut axially on the interior cylindrical member, the spiral slot configured to reduce friction between an outer surface of the interior cylindrical member and the inner surface of the exterior cylindrical member. In another embodiment, the first set of vitreous ports includes a first port and the second set of vitreous ports includes a second port. The first and second ports are substantially oppositely disposed on a circumferential plane that is perpendicular to the axis of the exterior cylindrical member.

In another embodiment, a method is disclosed that comprises inserting through a port in an eye an exterior cylindrical member having two substantially separately disposed sets of ports including a first plurality of ports and a second plurality of ports that is disposed separately from the first plurality of ports, receiving, during a vitrectomy, vitreous via the two substantially separately disposed sets of ports, the exterior cylindrical member having an open proximal end and a closed distal end, and the two oppositely disposed sets of ports being positioned adjacent to the closed distal end of the exterior cylindrical member; reciprocating an interior cylindrical member axially within the exterior cylindrical member, the interior cylindrical member having an open proximal end and an open distal end that is adjacent to the closed distal end of the exterior cylindrical member wherein a cutting edge is formed on said open distal end of the interior cylindrical member, the method further comprising cutting with the cutting edge vitreous that is received through the two oppositely disposed sets of ports as the interior cylindrical member reciprocates within the exterior cylindrical member.

In another embodiment, the open distal end of the interior cylindrical member is structured to have a funnel shape, and an exterior surface of the interior cylindrical member includes an electrolyte configured to harden the cutting edge of the interior cylindrical member. In another embodiment, the interior cylindrical member includes an axial spiral slot that reduces friction between an outer surface of interior cylindrical member and the inner surface of the exterior cylindrical member.

In a further embodiment, a system comprises an exterior cylindrical member having two substantially oppositely disposed ports including a first port and a second port that is substantially oppositely disposed from the first port, the two substantially oppositely disposed ports for receiving vitreous during a vitrectomy procedure, the exterior cylindrical member having an open proximal end and a closed distal end, and the two substantially oppositely disposed ports being positioned adjacent to the closed distal end of the exterior cylindrical member with an interior cylindrical member reciprocating axially within the exterior cylindrical member, the interior cylindrical member having an open proximal end and an open distal end that is adjacent to the closed distal end of the exterior cylindrical member wherein a cutting edge is formed on said open distal end of the interior cylindrical member with the cutting edge for cutting vitreous received through the two substantially oppositely disposed ports as the interior cylindrical member reciprocates within the exterior cylindrical member.

In another embodiment, the open distal end of the interior cylindrical member is structured to have a funnel shape. In a further embodiment, an electrolyte disposed on an exterior surface of the interior cylindrical member, the electrolyte configured to harden and sharpen the cutting edge of the interior cylindrical member. Further yet, the interior cylindrical member includes an axial spiral slot, the axial spiral slot configured to reduce friction between an outer surface of the interior cylindrical member and the inner surface of the exterior cylindrical member.

A further understanding of the nature and advantages of the present invention herein may be realized by reference to the remaining portions of the specification and the attached drawings. Further features and advantages of the present invention, as well as the structure and operation of various embodiments of the present invention, are described in detail below with respect to the accompanying drawings. In the drawings, the same reference numbers indicate identical or functionally similar elements.

### BRIEF DESCRIPTION OF THE DRAWINGS

FIG. 1 illustrates a front plan view of a human eye during vitrectomy surgery in accordance with an embodiment of the present invention.

FIG. 2 is a cross-sectional view of the human eye of FIG. 1.

FIG. 3 illustrates a cross sectional side view of a vitrectomy cutter according to an exemplary embodiment of the present invention.

FIG. 4 is a top plan view of the exterior cylindrical member of the vitrectomy cutter of FIG. 3.

FIG. 5 is a perspective view of the exterior cylindrical member of the vitrectomy cutter of FIG. 3.

FIG. 6 illustrates a vitrectomy cutter in accordance with another exemplary embodiment of the present invention.

FIG. 7 illustrates a vitrectomy cutter in accordance with another exemplary embodiment of the present invention.

FIG. 8 is a perspective view of the interior cylindrical member of FIG. 7.

FIG. 9 illustrates a side view of the interior cylindrical member of FIG.

FIG. 10 illustrates a top plan view of an exterior cylindrical member of a vitrectomy cutter according to another exemplary embodiment of this invention.

FIG. 11 illustrates a bottom plan view of the exterior cylindrical member of the vitrectomy cutter of FIG. 10.

FIG. 12 is a perspective view of the exterior cylindrical member of the vitrectomy cutter of FIG. 10 in accordance with an exemplary embodiment of the present invention.

### DETAILED DESCRIPTION OF THE INVENTION

Reference will now be made in detail to the embodiments of the invention, examples of which are illustrated in the accompanying drawings. While the invention will be described in conjunction with the one embodiment, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth to provide a thorough understanding of the present invention. However, it will be obvious to one of ordinary skill in the art that the present invention may be practiced without these specific details. In other instances, well-known methods, procedures, components, and circuits have not been described in detail as to not unnecessarily obscure aspects of the present invention.

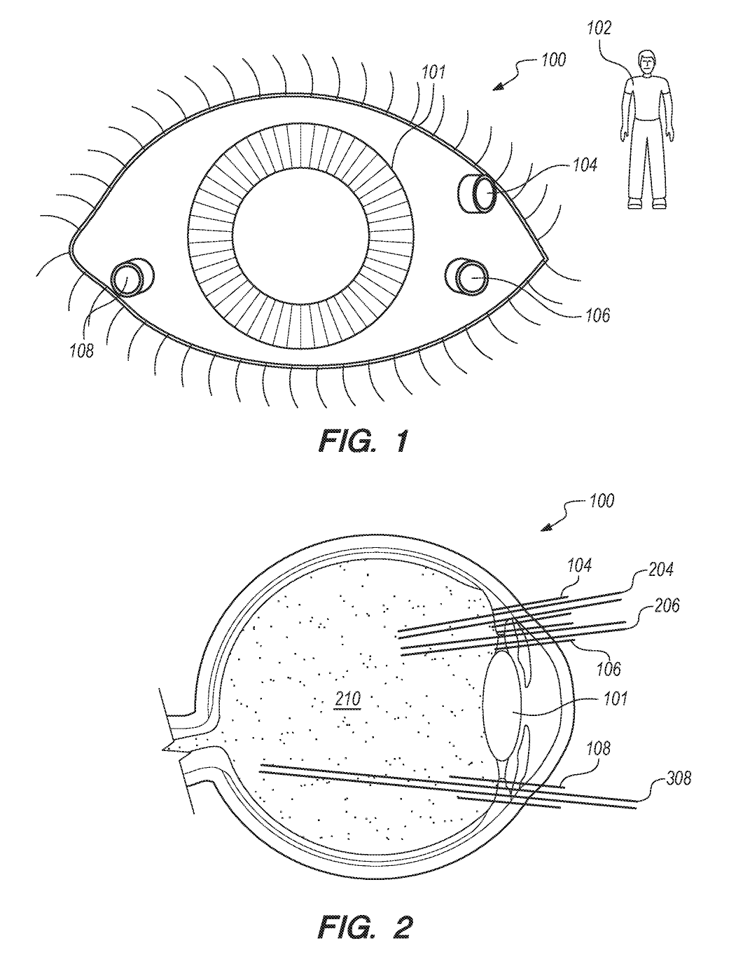

FIG. 1 illustrates a front plan view of human eye 100 during vitrectomy surgery in accordance with an embodiment of the present invention.

In FIG. 1, a user or eye surgeon 102 may perform vitrectomy on human eye 100 to rectify vision impairment such as that associated with retinal detachment (for example). This surgical procedure might specifically be performed to remove vitreous humor 210 (see FIG. 2) from human eye 100.

As shown in FIG. 1, eye surgeon 102 begins by inserting a number of ports 104, 106 and 108 adjacent to iris 101. Specifically, the inserted ports are light port 104, saline port 106 and vitrectomy cutter port 108. Here, each port is an entryway for inserting a surgical instrument into human eye 100 as further illustrated with reference to FIG. 2.

FIG. 2 is a cross-sectional view of human eye 100 illustrating surgery instruments inserted into light port 104, saline port 106 and vitrectomy cutter port 108 of FIG. 1. Here, eye surgeon 102 (of FIG. 1) passes an optical probe 204 through light port 104 into the interior of human eye 100 as shown. Optical probe 204 can then be employed to illuminate the interior of human eye 100 and maintain visibility as vitrectomy is performed. Eye surgeon 102 has the flexibility to move and redirect the light probe to the various areas of the eye interior as needed for illumination.

After insertion of optical probe 204, a saline tube 206 is then passed through saline port 106, the saline tube 206 permitting introduction of saline (or other comparable liquid or gaseous matter) into the eye, thus maintaining the eye's roundness as vitreous humor 210 is removed from human eye 100.

A vitrectomy cutter port 108 is also inserted into human eye 100. As implied by its name, vitrectomy cutter port 108 enables eye surgeon 102 to pass a vitrectomy cutter 308 through vitrectomy cutter port 108 to cut and aspirate vitreous humor 210 from human eye 100. In accordance with principles and precepts of the present invention, vitrectomy cutter 308, in one embodiment, can efficiently remove vitreous humor 210 (and vitreous humor base) while maintaining high performance without sacrificing stiffness or rigidity of the instrument.

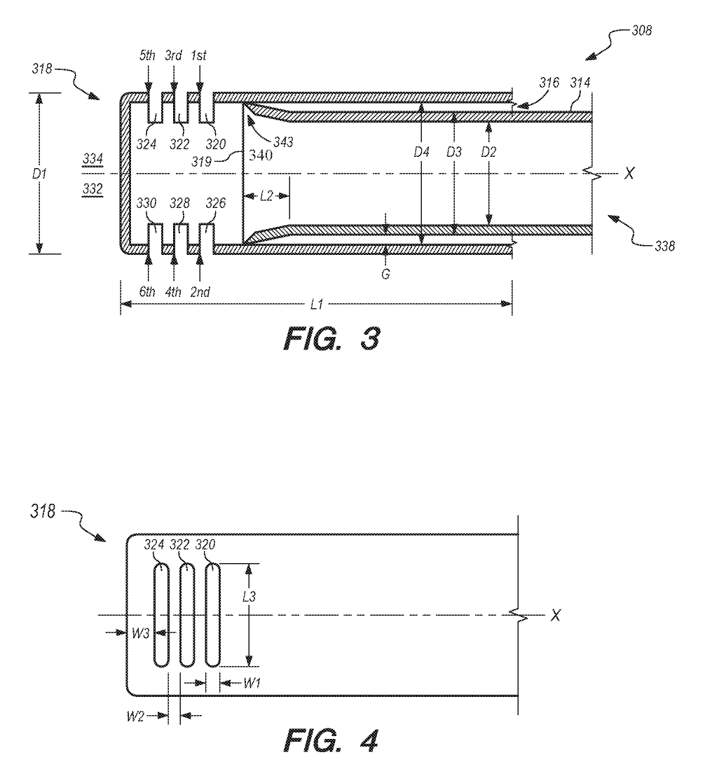

FIG. 3 illustrates a cross sectional side view of vitrectomy cutter 308 according to an exemplary embodiment of the present invention.

In FIG. 3, eye surgeon 102 may utilize vitrectomy cutter 308 for performing one or more intraocular surgical procedures. Such procedures may include vitrectomy for retinal eye surgery, macular hole, diabetic retinopathy, retinal detachment, uveitis and age-related macular degeneration, for example. Although not shown, it is contemplated that embodiments of the present invention can utilized for other surgery types for and beyond human eye applications.

In FIG. 3, among other components, vitrectomy cutter 308 comprises an exterior cylindrical member 312, and an interior cylindrical member 314 that reciprocates within said exterior cylindrical member 312. It is this exterior cylindrical member 312 that is in contact with vitrectomy cutter port 108 of FIG. 2 and vitreous humor 210 of FIG. 2 in the interior of human eye 100.

As shown in FIG. 3, exterior cylindrical member 312 has an open proximal end generally indicated as 316 and a closed distal end 318. Open proximal end 316 is generally left open to receive and cooperate with interior cylindrical member 314. Closed distal end 318, however, is a flat and smooth surface that closes off the instrument. This flat and smooth surface facilitates smooth movement of vitrectomy cutter 308 within human eye 100.

Here, exterior cylindrical member 312 might be made of stainless steel, high grade aluminum or other comparable material that can maintain stiffness/rigidity consistent with the spirit and scope of the present invention. In the embodiment of FIG. 3, exterior cylindrical member 312 has a diameter of D1 in the range of 0.025 inches. One skilled in the art will realize that the aforementioned and subsequent dimensions are exemplary and other dimensions consistent with the spirit and scope of the present invention may be utilized. Here, exterior cylindrical member 312 might have a standard axial length L1.

Referring to FIG. 3, exterior cylindrical member 312 further includes a plurality of slots that define multiple vitreous ports 320, 322, and 324. Each vitreous port 320, 322, 324 is an entryway that passes vitreous humor 210 (see FIG. 2) from human eye 210 into the interior of exterior cylindrical member 312 for cutting and removal by interior cylindrical member 314 as further described below.

Unlike traditional systems, the present embodiment includes at least three or more slots or vitreous ports 320, 322 and 324 on one side. The present embodiment also includes three or more vitreous ports 326, 328 and 330 that are separately or oppositely disposed. In this manner, additional vitreous humor not contemplated for removal by traditional systems can be drawn through the vitreous ports, cut and aspirated, thus enabling quicker and more efficient vitrectomy surgeries. The number of ports and rigidity are a fine balance; yet the embodiment of FIG. 3 attains increased efficiency without sacrificing rigidity of the instrument.

In FIG. 4, the top plan view of exterior cylindrical member 312 illustrating vitreous ports 320, 322 and 324 is shown. In FIG. 5, a perspective view of exterior cylindrical member 312 illustrating vitreous ports 320, 322 and 324 is also shown.

As can be seen in FIGS. 4 and 5, vitreous ports 320, 322 and 324 are identical and are formed on the periphery of exterior cylindrical member 312. Each of vitreous ports 320, 322 and 324 is adjacent to and parallel to the others. By parallelism, it is meant that each of vitreous ports 320, 322, 324 is aligned with the others having a direction that follows the curvature of exterior cylindrical member 312 around the longitudinal axis X of exterior cylindrical member 312 as seen more clearly in FIG. 5. This parallelism with at least three ports along the curvature and with oppositely disposed ports is an advantage because additional ports are added while maintaining structural integrity because the port depths (distance around the curvature between the end of the ports and the beginning of the next set of oppositely disposed port) can be controlled.

Here, each of vitreous ports 320, 322 and 324 has a length L3 0.022 inches and a width W1 of 0.005 inches. Each of vitreous ports 320, 322, 324 is spaced out from the others at a width W2 that is approximately 0.010 inches while the spacing W3 between vitreous port 324 and closed end 318 is about 0.010 inches. It can then be seen that unlike conventional vitrectomy systems, an embodiment of the present invention provides overall virtual port openings that are larger than conventional systems while maintaining the stiffness and rigidity of the vitrectomy cutter.

By increasing dimensions and the number of ports, additional vitreous can be allowed into the ports and cut such that a more efficient vitrectomy surgery may be performed. It is noted that increasing the number of ports and the size of each port is a delicate balance that has been realized by the present invention because while the number of ports and dimensions of each port have been increased, stiffness and rigidity have not been sacrificed by considering optimum placement, positioning, size and number of ports.

The exterior cylindrical member 312 maintains its stiffness or rigidity by defining and determining the appropriate number of ports and the optimum size for each of the ports. In one implementation, shown in FIGS. 3 and 4, each port is generally inline and extends as a plurality of slots that are perpendicular to axis X of the exterior cylindrical member 312.

Referring to FIG. 3, another advantage of the present invention is that exterior cylindrical member 312 further comprises another set of vitreous ports 326, 328, 330 that are separately and oppositely disposed from vitreous ports—320, 322, and 324. Vitreous ports 320, 322 and 324 and vitreous ports 326, 328 and 330 are separately and oppositely disposed because they are disposed on upper end 334 and lower end 324 respectively.

Thus, as further illustrated in FIG. 5, plane P extends axially through axis X to divide exterior cylindrical member 312 into equal halves namely upper end 334 and lower end 332. In one embodiment, vitreous port 326 and vitreous port 324 are said to be separately disposed from each other because they are disposed on separate upper and lower ends irrespective of the location of the ports in the upper and lower end.

In another embodiment, vitreous port 326 is oppositely disposed from vitreous port 320 because they are in opposite positions on the same plane in separate lower and upper ends. That is, vitreous port 326 and vitreous port 320 are substantially oppositely disposed because they are disposed at opposite positions on the circumference of a plane (not shown) that is cut perpendicular to axis X at the location of the two ports.

By having at least a second plurality of sets or ports that are disposed separately from the first plurality of ports, the present invention facilitates efficient vitrectomy surgeries because the amount of manipulation needed to turn vitreous ports toward vitreous humor is dramatically reduced.

As an example, in prior art systems that have only a single set of ports, once the vitreous that is facing the set of ports has been aspirated, it is necessary for the surgeon to then turn or manipulate the set of ports towards additional vitreous, thus causing unnecessary trauma to the patient. With embodiments of the present invention, such twisting and turning or manipulating of the vitrectomy cutter that can cause further trauma within human eye 100 is eliminated.

Referring now to FIG. 3, as previously noted, vitrectomy cutter 308 further includes interior cylindrical member 314 that reciprocates within the exterior cylindrical member 312 is a longitudinal direction (parallel to axis X). Interior cylindrical member 314 also has an open proximal end 338 and an open distal end 340. Open proximal end 338 and open distal end 340 are coupled via a bore 343.

Although not shown, open proximal end 338 is coupled to a vacuum or aspiration system (not shown) that aspirates cut vitreous humor 210 from open distal end 340 through bore 343 and/or open proximal end 338 for disposal. Similarly, although not shown, interior cylindrical member 314 is coupled to either a pneumatic or electrical driver (not shown) that reciprocates interior cylindrical member 314 backwards and forwards along the longitudinal axis X (FIG. 5). Those of ordinary skill in the art are familiar with such systems and they need not be described in detail.

In FIG. 3, interior cylindrical member 314 includes a cutting edge 319 formed on open distal end 340 of interior cylindrical member 314. Cutting edge 319 forms a circumferential cutting edge on open distal end 340. The edge is, itself, tapered so that it is sharp. Cutting edge 319 (along with open distal end 340) is also flared relative to the rest of the interior cylindrical member 314 as illustrated by the two arrows at the open distal end 340. That is, cutting edge 319 forms a funnel shape with respect to the rest of interior cylindrical member 314. In this manner, open distal end 340 is expanded, thus enhancing flow and aspiration of a larger amount of vitreous humor 210, which in turn facilitates faster and more efficient vitrectomy surgery.

As shown in FIG. 3, the interior diameter D2 of interior cylindrical member 314 may be 0.022 inches or 0.017 inches. As noted, the stated dimensions are exemplary. For example, D2 may also be 0.014 is 0.010 inches. It is further noted that dimensions may differ based on the gauge of the instrument. The different gauges might be 20 G, 23 G, 25 G or 27 G, for example. The exterior diameter D3 of interior cylindrical member 314 may be 0.028 inches, 0.020 inches, 0.014 inches or 0.012 inches, for example. Exemplary dimensions of the interior diameter D4 of exterior cylindrical member 312 may be 0.030 inches, 0.022 inches, 0.017 inches or 0.014 inches.

D4 and D3 are dimensioned such that a gap G (of about 0.002 inches) exists between the outer wall of interior cylindrical member 314 and the inner wall of exterior cylindrical member 312. The gap G reduces friction between the interior and exterior wall and facilitates smooth operation of the cylindrical members.

In operation, eye surgeon 102 begins by inserting vitrectomy cutter 308 through vitrectomy cutter port 108 of FIG. 2. Once the vitrectomy cutter 308 is inserted, eye surgeon 102 may activate an attached pneumatic or electrical driver to move interior cylindrical member 314 and its cutting edge 319 toward closed distal end 318 of exterior cylindrical member 312. Cutting edge 319 of interior cylindrical member 314 is disposed adjacent to closed distal end 318 of the exterior cylindrical member 312.

As cutting edge 319 approaches closed distal end 318, vitreous humor 210 that enters vitreous ports 320, 322, 324, 326, 328 and 330 is severed by cutting edge 319. Specifically, at a 1^{st} cutting point (vitreous port 320) and a 2^{nd} cutting point (vitreous port 326), the embodiment of the present invention employs cutting edge 319 to contemporaneously sever vitreous humor 210 at both cutting points. Conventional systems have one or more aggregated ports with cutting edges angled toward the aggregated ports to accomplish cutting. Such angled ports thus preclude the use of separated or oppositely disposed ports.

Unlike such traditional systems that employ aggregated ports with angled cutters to accomplish cutting, an embodiment of the present invention employs the flared cutting edge 319 that can contact all sides of the inner diameter of exterior cylindrical member 312. This ability to contact all sides of the inner diameter allows the present embodiment to employ separated and/or oppositely disposed vitreous ports 320, 322, 324, 326, 328 and 330.

After vitreous humor 210 is contemporaneously severed at the 1^{st} and 2^{nd} cutting points, cutting edge 319 then proceeds to the 3^{rd} and 4^{th} cutting points where vitreous humor 210 is again contemporaneously severed. Vitreous humor 210 is also contemporaneously severed at the 5^{th} (vitreous port 324) and 6^{th} (vitreous port 330). After cutting, all of the severed vitreous humor 210 is aspirated and removed via bore 343. Hence, converting targeted vitreous into tiny particles, which become easier to aspirate.

Depending upon the cut rate of the cutting console employed, the present embodiment also significantly increases the cutting rate CPM (cuts per minute) relative to conventional systems. In one embodiment, a cutting console of 5,000 CPM is used; thus, a cut rate of 30,000 CPM (5,000 cuts per port×6 ports) is realized. Where a cutting console of 8,000 CPM is used, the output rate of the present embodiment is 48,000 CPM (8,000 cuts per port×6 ports).

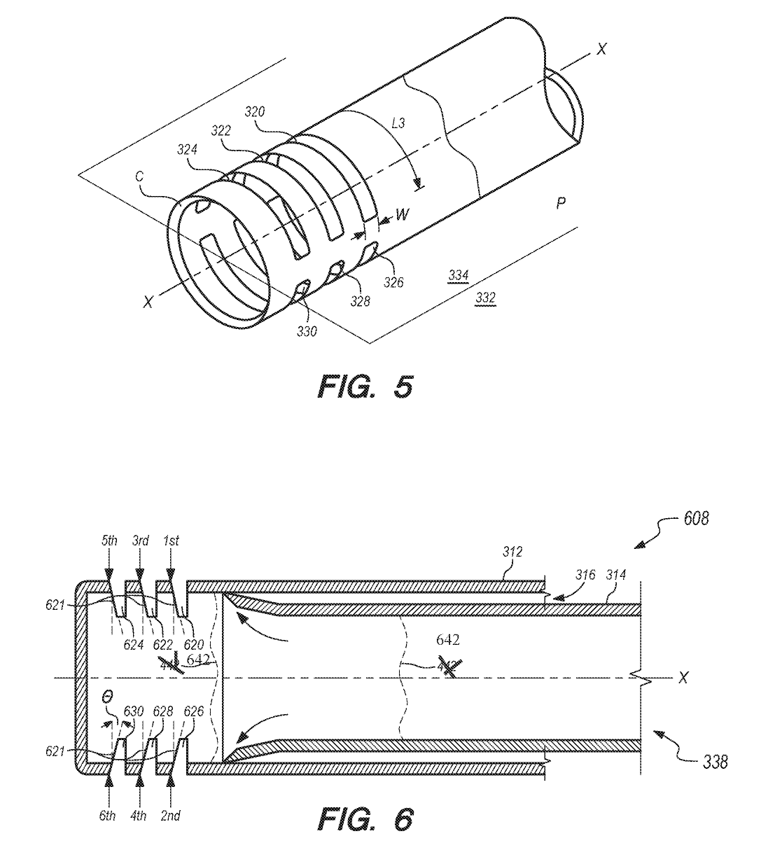

FIG. 6 illustrates a vitrectomy cutter 608 in accordance with an exemplary embodiment of the present invention.

Unlike the embodiment of FIG. 3 where vitrectomy cutter 308 has vitreous ports with essentially right angled vitreous ports, vitrectomy cutter 608 has vitreous ports 620, 622, 624, 626, 628, and 630, all of which have angled walls 621 at their 1^{st}, 2^{nd}, 3^{rd}, 4^{th}, 5^{th} and 6^{th} cutting points. Angled walls 621 function to provide more visibility during cutting, hence adds accuracy and improved cutting.

Another advantage of the present embodiment is that an electrolyte layer 642 may be deposited on the closed end area of exterior cylindrical member 312. Electrolyte layer 642 is also deposited on and about cutting edge 319 of interior cylindrical member 314 (FIG. 3). In this manner, electrolyte layer 642 can harden the areas of the cylindrical members to which the electrolyte layers are applied. Moreover, cutting edge 319 is also sharpened by electrolyte layer 642, and this sharpness will be maintained over time.

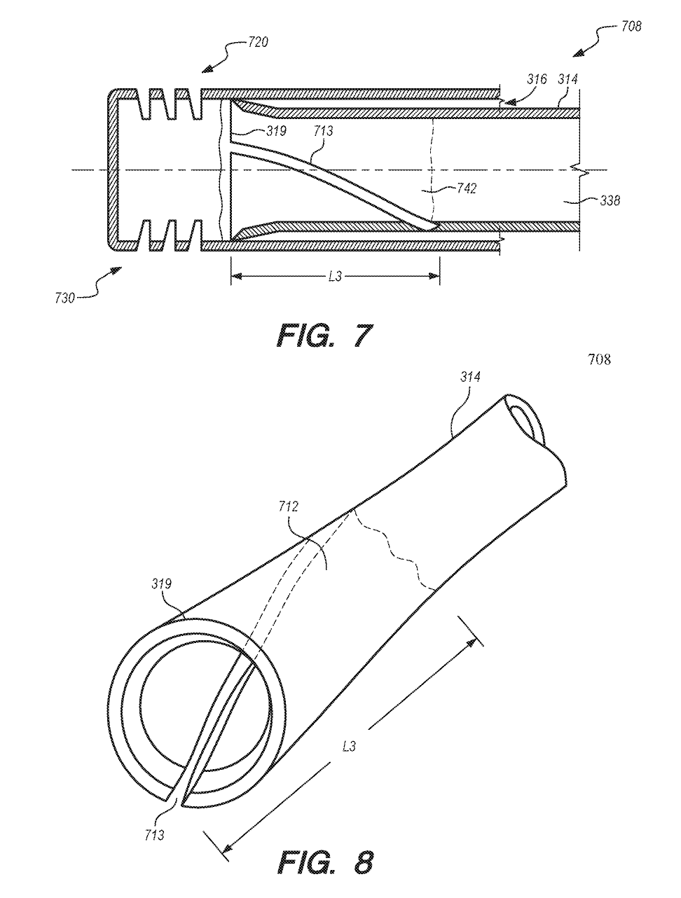

FIG. 7 illustrates vitrectomy cutter 708 in accordance with an exemplary embodiment of the present invention.

In FIG. 7, vitrectomy cutter 708 includes interior cylindrical member 314 as well as exterior cylindrical member 312 with angled ports 720 and 730 as in previous embodiments. However, vitrectomy cutter 708 further includes a spiral slot 713. Spiral slot 713 reduces friction between exterior cylindrical member 312 and interior cylindrical member 314 as the interior cylindrical member 314 reciprocates backwards and forwards within exterior cylindrical member 312.

As shown, spiral slot 713 extends from cutting edge 319 axially towards open proximal end 338 of interior cylindrical member 314. Specifically, spiral slot 713 may extend for a length L3 which may be 0.010 inches. Electrolyte layer 742 is also deposited on interior cylindrical member 314 for the length L3 of spiral slot 713.

FIG. 8 is a perspective view of vitrectomy cutter 708 of FIG. 7. As can be seen, the path for spiral slot 713 begins from cutting edge 319 and extends for length L3.

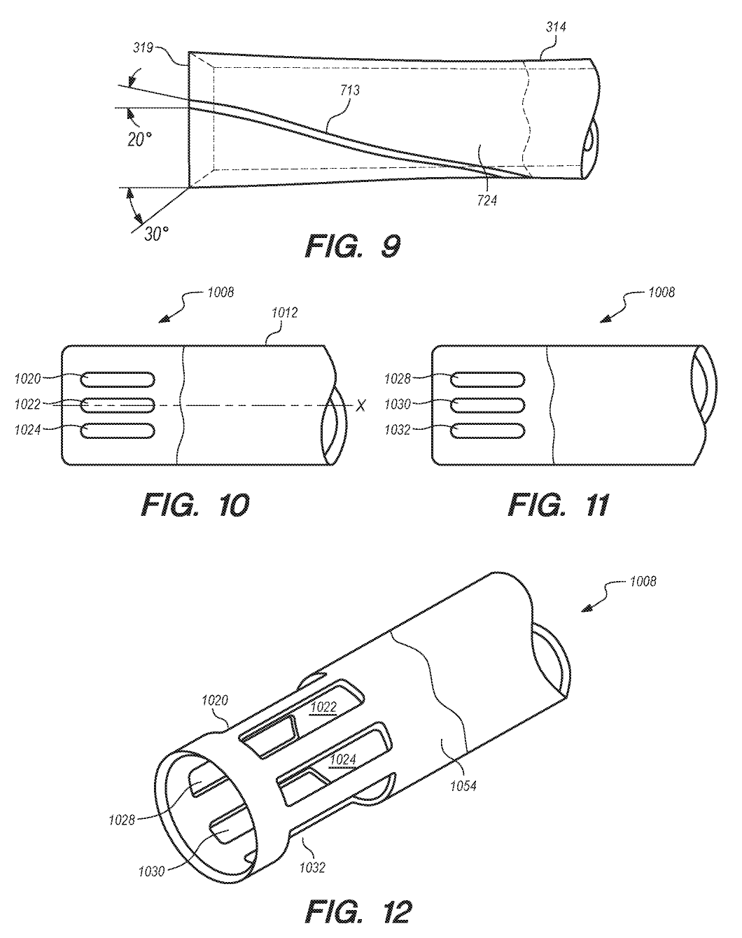

FIG. 9 illustrates a side view of interior cylindrical member 314 of FIG. 7. As can be seen in FIG. 9, spiral slot 713 is angled 20 degrees from the horizontal axis at cutting edge 319. Cutting edge 319 is itself tapered at an angle of 30 degrees from the horizontal axis to sharpen cutting edge 319.

FIG. 10 illustrates vitrectomy cutter 1008 according to an exemplary embodiment of this invention.

In FIG. 10, as can be seen, the vitreous ports 1020, 1022, and 1024 are inline or are horizontally disposed parallel to axis X of exterior cylindrical member 1012. Specifically, vitreous ports 1020, 1022, and 1024 are all disposed so that their length is along axis X. Although not shown, a plurality of oppositely disposed ports is below ports 1020, 1022, and 1024.

FIG. 11 illustrates a plan view of vitrectomy cutter 1008 showing a second side of exterior cylindrical member 1012 of FIG. 10. As can be seen, there are three additional ports 1028, 1030, and 1032 that are separately disposed from ports 1020, 1022, and 1024 as further illustrated in FIG. 12.

FIG. 12 is a perspective view of vitrectomy cutter 1008 showing vitreous ports 1020, 1022, and 1024 and vitreous ports 1028, 1030, and 1032. Electrolyte layer 1054 that is deposited around the vitreous ports is also shown.

While the above is a complete description of exemplary specific embodiments of the invention, additional embodiments are also possible. Thus, the above description should not be taken as limiting the scope of the invention, which is defined by the appended claims along with their full scope of equivalents.

I claim:

1. An apparatus comprising:
an exterior cylindrical member having two substantially oppositely disposed or separated sets of ports including a first plurality of ports, and a second plurality of ports that are disposed substantially oppositely from the first plurality of ports, the two substantially oppositely disposed sets of ports for receiving vitreous during a vitrectomy procedure, the exterior cylindrical member having an open proximal end and a closed distal end, and the two substantially oppositely disposed sets of ports being positioned adjacent to the closed distal end of the exterior cylindrical member; and
an interior cylindrical member reciprocating axially within the exterior cylindrical member, the interior cylindrical member having an open proximal end and an open distal end, with the open distal end of the interior cylindrical member being adjacent to the closed distal end of the exterior cylindrical member, wherein a cutting edge is formed on said open distal end of the interior cylindrical member, the cutting edge for cutting vitreous that is receivable through the two substantially oppositely disposed sets of ports as the interior cylindrical member reciprocates within the exterior cylindrical member.

2. The apparatus of claim 1 wherein the first plurality of ports is structured to have at least three ports.

3. The apparatus of claim 2 wherein the second plurality of ports is structured to have at least three ports.

4. The apparatus of claim 1 wherein the two oppositely disposed sets of ports are structured with at least six ports.

5. The apparatus of claim 1 wherein said cutting edge of the interior cylindrical member is flared to have a funnel shape.

6. The apparatus of claim 1 wherein said cutting edge is tapered from a proximal to distal end.

7. The apparatus of claim 1 wherein the first plurality of ports includes a first port and wherein the second plurality of ports includes a second port, wherein said first and second ports are substantially oppositely disposed on a circumferential plane perpendicular to the axis of the exterior cylindrical member.

8. The apparatus of claim 1 further comprising an electrolyte disposed on an exterior surface of the interior cylindrical member, the electrolyte configured to harden and sharpen the cutting edge of the interior cylindrical member.

9. The apparatus of claim 1 further comprising a spiral slot cut axially on the interior cylindrical member, the spiral slot configured to reduce friction between an outer surface of the interior cylindrical member and the inner surface of the exterior cylindrical member.

10. A method comprising:
inserting through a port in an eye, an exterior cylindrical member having two substantially separately disposed sets of ports including a first plurality of ports, and a second plurality of ports that is disposed separately from the first plurality of ports,
receiving, during a vitrectomy, vitreous via the two substantially separately disposed sets of ports, the exterior cylindrical member having an open proximal end and a closed distal end, and the two oppositely disposed sets of ports being positioned adjacent to the closed distal end of the exterior cylindrical member;
reciprocating an interior cylindrical member axially within the exterior cylindrical member, the interior cylindrical member having an open proximal end and an open distal end that is adjacent to the closed distal end of the exterior cylindrical member; wherein a cutting edge is formed on said open distal end of the interior cylindrical member; and
cutting, with the cutting edge, vitreous received through the two oppositely disposed sets of ports as the interior cylindrical member reciprocates within the exterior cylindrical member.

11. The method of claim 10 wherein the first plurality of ports is structured to have at least three ports.

12. The method of claim 10 wherein the second plurality of ports is structured to have at least three ports.

13. The method of claim 10 wherein said open distal end of the interior cylindrical member is structured to have a funnel shape.

14. The method of claim 10 further wherein an exterior surface of the interior cylindrical member includes an electrolyte configured to harden the cutting edge of the interior cylindrical member.

15. The method of claim 10 wherein the interior cylindrical member includes an axial spiral slot that reduces friction between an outer surface of interior cylindrical member and the inner surface of the exterior cylindrical member.

16. A system comprising:
an exterior cylindrical member having two substantially oppositely disposed ports including a first port and a second port that is substantially oppositely disposed from the first port, the two substantially oppositely disposed ports for receiving vitreous during a vitrectomy procedure, the exterior cylindrical member having an open proximal end and a closed distal end, and the two substantially oppositely disposed ports being positioned adjacent to the closed distal end of the exterior cylindrical member; and
an interior cylindrical member reciprocating axially within the exterior cylindrical member, the interior cylindrical member having an open proximal end and an open distal end that is adjacent to the closed distal end of the exterior cylindrical member; wherein a cutting edge is formed on said open distal end of the interior cylindrical member, the cutting edge for cutting vitreous received through the two substantially oppositely disposed ports as the interior cylindrical member reciprocates within the exterior cylindrical member.

17. The system of claim 16 wherein said open distal end of the interior cylindrical member is structured to have a funnel shape.

18. The system of claim 16 further comprising an electrolyte disposed on an exterior surface of the interior cylindrical member, the electrolyte configured to harden and sharpen the cutting edge of the interior cylindrical member.

19. The system of claim 16 wherein the interior cylindrical member includes an axial spiral slot, the axial spiral slot configured to reduce friction between an outer surface of interior cylindrical member and the inner surface of the exterior cylindrical member.
